(19) United States
(12) Patent Application Publication
(10) Pub. No.: US 2008/0058740 A1
(43) Pub. Date: Mar. 6, 2008

(54) Sensing article for a home automation network

(76) Inventors: Shawn J. Sullivan, Neenah, WI (US); Gary A. Clement, Menasha, WI (US); Sridhar Ranganathan, Suwanee, GA (US); Thomas M. Ales, Neenah, WI (US); Andrew M. Long, Appleton, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/511,572

(22) Filed: Aug. 29, 2006

(51) Int. Cl. A61F 13/15

(52) U.S. Cl. 604/361; 340/286.01

(57) ABSTRACT

Provided is a sensing system for use in conjunction with a home automation network controlling an environmental variable in a home, the system including a transmitter adapted to be used with an absorbent article having a sensor, wherein the transmitter is adapted to communicate with the sensor and directly with the home automation network. Also provided is a physiological event notification system including an absorbent article including a physiological event sensor; a home automation network controlling an environmental variable in a home; and a transmitter associable with the absorbent article and in signal communication with the physiological event sensor and with the home automation network.

### BACKGROUND OF THE INVENTION

Absorbent articles such as diapers, training pants, incontinence products, feminine hygiene products, swim undergarments, and the like conventionally include a liquid permeable body-side liner, a liquid impermeable outer cover, and an absorbent core. The absorbent core is typically located in between the outer cover and the liner for taking in and retaining liquids (e.g., urine) exuded by the wearer.

Many absorbent articles have been adapted for use in a training program, such as toilet training or enuresis control, or to provide indication of various medical, physical, or other conditions. Accordingly, various types of sensors and indicators, including moisture or wetness indicators, have been suggested for use in absorbent articles. Wetness indicators, for example, may include alarm devices that are designed to assist parents or attendants to identify a wet diaper condition quickly upon insult. The devices produce either a visual or an audible signal.

Visual or audible signals may not always be the appropriate or best means for communicating with a caregiver. In some aspects of the present invention, various sensors have been included in absorbent articles, where the sensors may communicate test results, indications, or other data to a user or a caregiver via a transmitter to a home automation network. In these aspects of the present invention, although the absorbent articles may be disposable, the transmitters typically are not. Thus, the transmitters are intended to be removed from the article and reattached to a subsequent article.

Home automation is a growing market. Leading manufacturers of electronic controls have been producing home automation components such as remote control light switches for years. Traditionally, home automation products have addressed timers, lighting products, remote switches, automatic pet feeders, motion detectors, HVAC controls, and similar non-critical components. New technologies have emerged that have improved the robustness and reliability of these components such that applications for health and hygiene can now be considered.

Taking care of one's family is of utmost importance to caregivers. A low cost scalable home automation infrastructure provides the backbone for a smart home to assist caregivers in their duties. The home automation infrastructure also provides a much needed solution to ensure that the caregiver can provide the best care.

The health and hygiene of sensing article home users may be improved by leveraging existing home automation technologies. As sensors begin to be included in absorbent and other articles such as training pants and adult care products, these articles can be integrated with home automation network technologies to improve the caregiver experience. By providing continuous monitoring of health and hygiene products though wired, wireless, and hybrid home networking technologies, the caregiver can be assured that they are giving the best possible care to those in their charge, while relieving them of some of the burden of patient attention.

### SUMMARY OF THE INVENTION

As caregivers undertake training, indication, and/or notification programs to address toilet training, enuresis control, incontinence monitoring, or condition monitoring, which apply across demographics, those caregivers would benefit by gaining greater access to products that are specifically designed to enhance the convenience, interest, and enjoyment of the products and thus the effectiveness of such programs.

The invention described herein solves the problems described above and provides an increase in convenience and efficacy in using wetness and other sensors in absorbent articles by increasing the convenience with which indications may be communicated. In general, the present disclosure is directed to sensing absorbent articles with easy-to-use transmitters. The transmitter, for instance, may be configured to indicate to a user that a body fluid is present in the sensing absorbent article.

For example, in one aspect of the present invention, the invention includes a sensing system for use in conjunction with a home automation network controlling an environmental variable in a home, the system including a transmitter adapted to be used with an absorbent article having a sensor, wherein the transmitter is adapted to communicate with the sensor and directly with the home automation network.

In another aspect of the present invention, the invention includes a sensing system for use in conjunction with a home automation network controlling an environmental variable in a home, the system including an absorbent article including a sensor and a transmitter, wherein the transmitter is adapted to communicate with the sensor and directly with the home automation network.

In another aspect of the present invention, the invention includes a physiological event notification system including an absorbent article including a physiological event sensor; a home automation network controlling an environmental variable in a home; and a transmitter associable with the absorbent article and in signal communication with the physiological event sensor and with the home automation network.

In another aspect of the present invention, the invention includes a method for benefiting a caregiver, the method including providing an absorbent article including a biological sensor and a transmitter, wherein the transmitter is adapted to communicate with the biological sensor and directly with a home automation network; and providing instructions to the caregiver with respect to responding to the home automation network once the home automation network has received a communication from the transmitter.

In another aspect of the present invention, the invention includes a sensing absorbent article system including an absorbent article having a liner, an outer cover, and an absorbent structure positioned between the liner and the outer cover, the absorbent article including a sensing means; and a transmitter capable of communicating with the sensing means and a home automation network.

### BRIEF DESCRIPTION OF THE DRAWINGS

The foregoing and other features and aspects of the present invention and the manner of attaining them will become more apparent, and the invention itself will be better understood by reference to the following description, appended claims and accompanying drawings, where:

FIG. 1 is a perspective view of an absorbent article of the present invention; and

FIG. 2 is a perspective view of another aspect of the absorbent article illustrated in FIG. 1.

Repeat use of reference characters in the present specification and drawings is intended to represent the same or analogous features or elements of the present invention. The drawings are representational and are not necessarily drawn to scale. Certain proportions thereof may be exaggerated, while others may be minimized.

### DETAILED DESCRIPTION OF THE INVENTION

It is to be understood by one of ordinary skill in the art that the present discussion is a description of exemplary aspects of the present invention only, and is not intended as limiting the broader aspects of the present invention.

The present disclosure is generally directed to sensing absorbent articles adapted to be attached to or include a transmitter, where the transmitter may be configured to communicate a body condition and/or the presence of a body fluid in the absorbent article, or other changes in the condition of the product or wearer. The absorbent article may be, for instance, a diaper, a training pant, a pre-fastened pant, a swimming pant, an incontinence product, a feminine hygiene product, a medical garment, a bandage, or any other suitable article.

The invention described herein may be used with any type of sensing article. In one type of sensing article used as a non-limiting example herein, a wetness sensing article may include an open circuit that becomes closed when a conductive fluid, such as a body fluid, is present between a pair of conductive leads. Alternatively, the sensing article may include a sensor to detect a specific substance or condition such as pH. Generally, the sensing article containing the sensor is disposable, meaning that it is designed to be discarded after a limited use rather than being laundered or otherwise restored for reuse. Part or all of the sensing article may be reusable. The sensor itself may be disposable, reusable, or some combination of the two.

A transmitter associated with the sensing article interprets the sensor detection and communicates with a home automation network, which in turn uses such communication as a basis for changing a home environment variable or for notifying a resident of the home or anyone else associated with the home automation network in some way.

The uniqueness of the present invention is its use of home automation technologies to integrate health and hygiene sensors into the home. This invention provides for a relatively low cost scalable system that has as much household coverage or as many nodes as the user desires to install. This system provides the infrastructure for remote sensors and monitoring by a caregiver for infants, children, elderly, and other uniquely challenged persons requiring monitoring.

Various home automation technologies exist that could be used to integrate health and hygiene products into the home. X10, Insteon, ZigBee, Zwave, CE bus, structured home wiring, and Echelon are examples of systems into which consumer health and hygiene products can be integrated. These technologies can be leveraged to provide affordable mesh networking of the home to monitor, record, track, and annunciate.

A home automation network 10 employs automation techniques to control the comfort and security of the residents of a home or other residence. The home automation network 10 controls home environment variables such as HVAC including temperature and humidity control; lighting; physical security including surveillance and the control of doors, windows, and shutters; sound including home entertainment systems; plant watering; pet feeding; appliances; intercoms; sprinklers; and any other variables that may be controlled based on time or other factors. As a part of such control, the home automation system can be configured to detect various conditions such as time; temperature; catastrophe detection including smoke, fire, and water; the presence of residents and/or intruders; and an inventory of household goods and can be configured to respond to any or all of these.

For example, a home automation network 10 may detect the entry of a home resident and respond by turning on air conditioning, lighting, and music, by turning off intruder detection in living areas of the home, and by surveying the contents of a pantry and generating a grocery shopping list to replenish missing items. The home automation network 10 may also then announce through an intercom or other system that a certain number of telephone messages were received, the current temperature of the home and of the outside, and any other relevant information.

A key component of most home automation systems is a human interface. A home automation system typically includes at least one display that may indicate to home residents the status of various components, the conditions of various home environment variables, and/or any other conditions or variables that the home automation network 10 monitors or controls. The human interface may also include light and/or sound indicators such as a fire alarm. In addition, the home automation network 10 may communicate with others outside the home such as emergency services, or with a computer or computer network within or outside the home.

The home automation network 10 may also communicate with the internet or other remote devices for notification to the caregiver or other suitable party as described in co-pending and co-assigned U.S. patent application Ser. No. 10/277,170, filed on Oct. 21, 2002 by Lindsay et al. and titled “Healthcare Networks With Biosensors,” which is incorporated herein by reference to the extent it is consistent (i.e., not in conflict) herewith

A home automation network 10 may be installed during construction of the home, or may be retrofitted in an existing home. Potential difficulties in running wiring in an existing home may be overcome using wireless and power line protocols such as INSTEON, X10, UPB, and Z-Wave.

INSTEON automation system is a wireless home-control networking technology that simply, affordably, and reliably integrates systems in the home for improved comfort, safety, convenience, and value (see www.insteon.org). INSTEON automation system, invented by SmartLabs Technology, is a robust, redundant dual-mesh network that combines wireless radio frequency (RF) with the home's existing electrical wiring. INSTEON automation system is less susceptible than other single band networks to the kind of interference and noise commonly encountered within the home.

INSTEON automation system leverages digital technology to create a true peer-to-peer mesh network. Because all INSTEON automation system devices are peers, they do not require network supervision, so complex network controllers and routing tables are not required. Building on a peer-to-peer mesh network foundation, INSTEON automation system confirms every message to insure that messages are received error-free, and automatically retransmits corrupted messages. Because every INSTEON automation system device acts as a two-way repeater, the INSTEON automation system becomes stronger and more reliable as it grows.

INSTEON automation system uses advanced digital signal processing to encode and transmit messages, enabling rapid transmission of control data between INSTEON automation system devices. Individual INSTEON automation system messages can also carry up to 14 bytes of arbitrary user data to support unlimited home-control applications from developers.

X10 is an industry standard for communication among devices used for home automation. It primarily uses power line wiring for signaling and control, where the signals involve brief radio frequency bursts representing digital information. A radio based transport is also defined.

X10 was developed in 1975 by Pico Electronics of Glenrothes, Scotland, in order to allow remote control of home devices and appliances. It was the first domotic technology and remains the most widely available.

Household electrical wiring is used to send digital data between X10 devices. This digital data is encoded onto a 120 kHz carrier which is transmitted as bursts during the relatively quiet zero crossings of the 50 or 60 Hz AC alternating current waveform. One bit is transmitted at each zero crossing.

The digital data consists of an address and a command sent from a controller to a controlled device. More advanced controllers can also query equally advanced devices to respond with their status. This status may be as simple as “off” or “on”, or the current dimming level, or even the temperature or other sensor reading. Devices usually plug into the wall where a lamp, television, or other household appliance plugs in; however some built-in controllers are also available for wall switches and ceiling fixtures.

The relatively-high-frequency carrier frequency carrying the signal cannot pass through a power transformer or across the phases of a multiphase system. In addition, because the signals are timed to coincide with the zero crossings of the voltage waveform, they would not be timed correctly to be coupled from phase-to-phase in a three-phase power system. For split phase systems, the signal can be passively coupled from phase-to-phase using a passive capacitor, but for three phase systems or where the capacitor provides insufficient coupling, an active X10 repeater is sometimes used.

Whether using power line or radio communications, packets transmitted using the X10 control protocol consist of a four bit “house code” followed by one or more four bit “unit codes,” finally followed by a four bit “command.” For the convenience of the users setting up the system, the four bit house code is labeled as one of the letters A through P while the four bit unit code is labeled as a number 1 through 16.When the system is installed, each controlled device is configured to respond to one of the 256 possible addresses (16 house codes*16 unit codes) and it will then only react to those commands specifically addressed to it.

In use, the protocol may transmit a message that says: “select house code A,” “select unit 3,” and “turn on” and the unit set to address “A3” will turn on its device. Several units can be addressed before giving the command, allowing the command to affect several units simultaneously. For example, “select house code A,” “select unit 3,” “select unit 5,” “select unit 4,” and finally, “turn on.” This will cause units A3, A4, and A5 to all turn on.

Note that there is no restriction (except possibly consideration of the neighbors) that prevents using more than one house code within a single house. The “all lights on” command and “all units off” commands will only affect a single house code, so an installation using multiple house codes effectively has the devices divided into separate zones.

Power line protocol physical-layer details: In the 60 Hz AC power flow, a Binary Digit (bit) 1 is represented by a 1 millisecond burst of 120 kHz at the zero crossing point (0-o, but certainly within 200 microseconds of the zero crossing point), immediately followed by the absence of a pulse. And a Binary 0 by the absence of 120 kHz at the zero crossing points (pulse), immediately followed by the presence of a pulse. All messages are sent twice to reduce false signaling. After allowing for retransmission, line control, etc, data rates are around 20 bit/s, making X10 data transmission so slow that the technology is confined to turning devices on and off or other very simple operations.

In order to provide a predictable start point, every data frame transmitted always begin with a start code of “pulse,” “pulse,” “pulse,” “absence of a pulse” (or 1110). Immediately after the start code, a letter code/house code (A-P) is sent and after the letter code, comes a function code. Function codes may specify a unit number code (1-16) or an actual command code, the selection between the two modes being determined by the last bit where 0=unit number and 1=command). One start code, one letter code, and one function code is known as an X10 frame and represent the minimum components of a valid X10 data packet.

Each signal is also sent two times to make sure the receivers understand it over the “noise” of the power lines (for purposes of redundancy, reliability, and to accommodate line repeaters).

Whenever the data changes from one address to another address, from an address to a command, or from one command to another command, the data frames must be separated by at least 6 clear zero crossings (or “000000”). The sequence of six “zeros” resets the shift registers that decode the received data packets.

The radio protocol: To allow the operation of wireless keypads, remote switches, and the like, a radio protocol is also defined. Operating at a frequency of 310 MHz in the U.S. and a different frequency in the rest of the world, the wireless devices send data packets that are very similar to ordinary X10 power line control packets. A radio receiver then provides a bridge which translates these radio packets to ordinary X10 power line control packets.

Device modules: Depending on the load that is to be controlled, different modules must be used. For incandescent lamp loads, a lamp module or wall switch module can be used. These modules switch the power using a triac solid-state switch and are also capable of dimming the lamp load. Lamp modules are silent in operation. They are generally rated to control loads that range from approximately 40 watts to 500 watts.

For loads other than incandescent lamps (for example, fluorescent lamps, high-intensity discharge lamps, and electrical appliances), the logic in the lamp module is unsuitable and an appliance module must be used instead. These modules switch the power using an impulse relay. In the U.S., these modules are generally rated to control loads that range from very little current up to 15 amperes.

Many device modules offer a feature called local control. If the module is switched off, operating the power switch on the lamp or appliance will cause the module to turn on. In this way, a lamp can still be lighted or a coffee pot turned on without the need to walk over to the X10 controller. Wall switch modules may not offer this feature.

Some wall switch modules offer a feature called local dimming. Ordinarily, the local pushbutton of a wall switch module simply offers on/off control with no possibility of locally dimming the controlled lamp. But if local dimming is offered, then holding down the push button will cause the lamp to cycle through its brightness range.

Higher end modules have more advanced features such as programmable on levels, customizable fade rates, the ability to transmit commands when used (referred to as 2-way devices), and scene support.

The circuit contained within the sensing articles of the present disclosure is configured to be attached to a transmitter. The transmitter can provide power to the circuit while also including some type of audible, visible and/or electromagnetic signal that indicates to the user the presence of a body fluid. Although the sensing article may itself be disposable, the transmitter may be reusable from article to article. In this regard, the present disclosure is particularly directed to different types of attachment mechanisms that allow easy connection between the circuit in the sensing article and the transmitter.

As described above, the circuit in combination with the transmitter may be configured to indicate the presence of a body fluid contained within the sensing article. The particular targeted body fluid may vary depending upon the particular type of sensing article and the desired application. For instance, in one aspect of the present invention, the sensing article comprises a diaper, a training pant, or the like and the transmitter is configured to indicate the presence of urine. Alternatively, the transmitter may be configured to indicate the presence of a metabolite that would indicate the presence of a diaper rash. For adult incontinence products and feminine hygiene products, on the other hand, the transmitter may be configured to indicate the presence of a yeast or of a particular constituent in urine or menses, such as a polysaccharide.

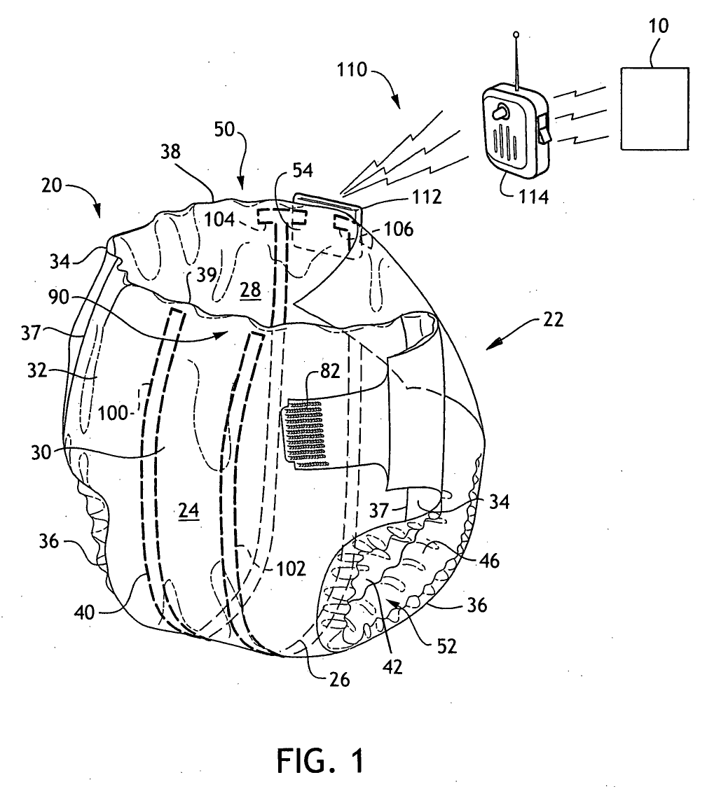

Referring to FIG. 1, for non-limiting exemplary purposes, a sensing article 20 is shown. All or part of the sensing article 20 may be disposable or reusable. It is understood that the present invention is suitable for use with various other sensing articles intended for personal wear, including but not limited to diapers, training pants, swim pants, feminine hygiene products, incontinence products, medical garments, surgical pads and bandages, other personal care or health care garments, and the like without departing from the scope of the present invention.

By way of illustration only, various materials and methods for constructing sensing articles such as the sensing article 20 of the various aspects of the present invention are disclosed in U.S. Pat. No. 4,798,603 issued Jan. 17, 1989, to Meyer et al.; U.S. Pat. No. 5,176,672 issued Jan. 5, 1993, to Bruemmer et al., U.S. Pat. No. 5,509,915 issued Apr. 23, 1996 to Hanson et al., U.S. Pat. No. 5,993,433 issued Nov. 30, 1999 to St. Louis et al., and U.S. Pat. No. 6,248,097 issued Jun. 19, 2001 to Beitz et al., PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent they are consistent (i.e., not in conflict) herewith.

A sensing article 20 is representatively illustrated in FIG. 1 in a partially-fastened condition.

The sensing article 20 defines a pair of longitudinal end regions, otherwise referred to herein as a front region 22 and a back region 24, and a center region, otherwise referred to herein as a crotch region 26, extending longitudinally between and interconnecting the front and back regions 22, 24. The sensing article 20 also defines an inner surface 28 adapted in use (e.g., positioned relative to the other components of the article 20) to be disposed toward the wearer, and an outer surface 30 opposite the inner surface. The front and back regions 22, 24 are those portions of the sensing article 20, which when worn, wholly or partially cover or encircle the waist or mid-lower torso of the wearer. The crotch region 26 generally is that portion of the sensing article 20 which, when worn, is positioned between the legs of the wearer and covers the lower torso and crotch of the wearer. The sensing article 20 has a pair of laterally opposite side edges 36 and a pair of longitudinally opposite waist edges, respectively designated front waist edge 38 and back waist edge 39.

The illustrated sensing article 20 includes a chassis 32 that, in this aspect of the present invention, encompasses the front region 22, the back region 24, and the crotch region 26. The chassis 32 includes an outer cover 40 and a bodyside liner 42 that may be joined to the outer cover 40 in a superimposed relation therewith by adhesives, ultrasonic bonds, thermal bonds or other conventional techniques. The liner 42 can be generally adapted, i.e., positioned relative to the other components of the article 20, to be disposed toward the wearer's skin during wear of the sensing article. The chassis 32 may further include an absorbent structure (not shown) disposed between the outer cover 40 and the bodyside liner 42 for absorbing liquid body exudates exuded by the wearer, and may further include a pair of containment flaps 46 secured to the bodyside liner 42 for inhibiting the lateral flow of body exudates. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the sensing article 20 may also suitably include leg elastic members (not shown), as are known to those skilled in the art.

In some aspects of the present invention, the sensing article 20 may further include a surge management layer (not shown) which may be optionally located adjacent the absorbent structure 44 and attached to various components in the article 20 such as the absorbent structure 44 or the bodyside liner 42 by methods known in the art, such as by using an adhesive. A surge management layer helps to decelerate and diffuse surges or gushes of liquid that may be rapidly introduced into the absorbent structure of the article. Desirably, the surge management layer can rapidly accept and temporarily hold the liquid prior to releasing the liquid into the storage or retention portions of the absorbent structure. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein to the extent they are consistent (i.e., not in conflict) herewith.

As shown in FIG. 1, the sensing article 20 further includes a pair of opposing elastic side panels 34 that are attached to the back region of the chassis 32. As shown particularly in FIG. 1, the side panels 34 may be stretched around the waist and/or hips of a wearer to secure the garment in place. The elastic side panels are attached to the chassis along a pair of opposing longitudinal edges 37. The side panels 34 may be attached or bonded to the chassis 32 using any suitable bonding technique. For instance, the side panels 34 may be joined to the chassis by adhesives, ultrasonic bonds, thermal bonds, or other conventional techniques. Ultimately, the side panels 34 are generally aligned with a waist region 90 of the chassis.

In an alternative aspect of the present invention, the elastic side panels may also be integrally formed with the chassis 32. For instance, the sidepanels 34mmay comprise an extension of the bodyside liner 42, of the outer cover 40, or of both the bodyside liner 42 and the outer cover 40.

The side panels 34 are connected to the back region of the sensing article 20 and extend over the front region of the article when securing the article in place on a user. It should be understood, however, that the side panels 34 may alternatively be connected to the front region of the article 20 and extend over the back region when the article is donned.

With the sensing article 20 in the fastened position as partially illustrated in FIG. 1, the elastic side panels 34 may be connected by a fastening system 82 to define a 3-dimensional sensing article configuration having a waist opening 50 and a pair of leg openings 52. The waist opening 50 of the article 20 is defined by the waist edges 38 and 39 which encircle the waist of the wearer.

In the aspects of the present invention shown in the figures, the side panels are releasably attachable to the front region 22 of the article 20 by the fastening system 82. It should be understood, however, that in other aspects of the present invention the side panels may be permanently joined to the chassis 32 at each end. The side panels may be permanently bonded together, for instance, when forming a training pant or absorbent swimwear.

The fastening system 82 may include laterally opposite first fastening components adapted for refastenable engagement to corresponding second fastening components. The fastening system 82 may include any refastenable fasteners suitable for sensing articles, such as adhesive fasteners, cohesive fasteners, mechanical fasteners, or the like. In particular aspects, the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like. Suitable fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

In addition to possibly having elastic side panels, the sensing article 20 may include various waist elastic members for providing elasticity around the waist opening. For example, as shown in the figures, the sensing article 20 can include a front waist elastic member 54 and/or a back waist elastic member (not shown).

In one aspect of the present invention, a body fluid indicating system takes the form of the wetness indicating system described herein. In this regard, as shown in FIG. 1, the sensing article 20 includes a first conductive element 100 spaced from a second conductive element 102. In this aspect of the present invention, the conductive elements extend from the front region 22 of the sensing article to the back region 24 without intersecting. The conductive elements 100 and 102 can comprise any suitable conductive material, such as a conductive thread or a conductive foil for example include 112-S silver metallic conductive paste (ink) from Electroscience Laboratories, Inc. and conductive foil described in U.S. Pat. No. 6,417,455 issued Jul. 9, 2002 to Zein et al. The first conductive element 100 may not intersect the second conductive element 102 in order to form an open circuit that may be closed, for instance, when a conductive fluid is positioned in between the conductive elements. In other aspects of the present invention, however, the first conductive element 100 and the second conductive element 102 may be connected to a sensor within the chassis. The sensor may be used to sense changes in temperature or may be used to sense the presence of a particular substance, such as a metabolite.

In the aspect of the present invention shown in FIG. 1, the conductive elements 100, 102 extend the entire length of the sensing article 20. It should be understood, however, that in other aspects of the present invention the conductive elements may extend only to the crotch region 26 or may extend to any particular place in the sensing article where a body fluid is intended to be sensed. In another aspect of the present invention, the conductive elements 100, 102 may be adapted to act as an antenna as well to increase the range of a transmitter connected to the conductive elements 100, 102, such as that described in U.S. Pat. No. 4,356,818, issued on Nov. 2, 1982 to Macias et al., and U.S. Pat. No. 6,097,297 issued on Aug. 1, 2000 to Fard.

The conductive elements 100, 102 may be incorporated into the chassis 32 at any suitable location as long as the conductive elements are positioned so as to contact a body fluid that is absorbed by the sensing article 20. In this regard, the conductive elements 100, 102 generally lie inside the outer cover 40. In fact, in one aspect of the present invention, the conductive elements 100, 102 may be attached or laminated to the inside surface of the outer cover 40 that faces the absorbent structure. Alternatively, however, the conductive elements 100, 102 may be positioned on the absorbent structure or positioned on the liner 42.

The conductive elements 100, 102 may be connected directly to a transmitter, either through direct or indirect contact. The first conductive element 100 may be attached to a first conductive pad member 104, while the second conductive element 102 may be connected to a second conductive pad member 106. The pad members 104, 106 may be provided for making a reliable connection between the open circuit formed by the conductive elements to a transmitter that is intended to be installed on the chassis by the consumer or manufacturer. The pad members 104, 106 may create a zone for connecting the transmitter and the conductive leads or elements.

The conductive pad members 104, 106 may have a relatively large surface area in relation to the conductive elements 100, 102. For example, the conductive pad members 104, 106 may have a surface area of at least 0.5 cm², at least 1 cm², at least 2 cm², and, in another aspect of the present invention, at least 3 cm². For instance, in another aspect of the present invention, the surface area of each pad member may be from about 2 cm² to about 4 cm².

The position of the conductive pad members 104, 106 on the sensing article 20 can vary depending upon where it is desired to mount the transmitter. For instance, in FIG. 1, the conductive pad members 104, 106 are positioned in the front region 22 along the waist opening of the article. In another aspect of the present invention that is not shown, the conductive pad members 104, 106 are positioned in the back region 24 along the waist opening of the article. It should be appreciated, however, that in other aspects of the present invention, the sensing article 20 may include conductive pad members being positioned at each end of each conductive element 100, 102. In still other aspects of the present invention, it should be understood that the pad members may be located along the side of the article or toward the crotch region of the article.

The position of the conductive pad members 104, 106 within the multiple layers of the chassis 32 may also vary depending upon where it is desired to connect the transmitter and the type of attachment mechanism used to make a connection with the transmitter. As described above, the pad members 104, 106 are electrically connected to the conductive elements 100, 102. Thus, in one aspect of the present invention, the pad members 104, 106 are positioned below (toward the body side) at least one layer of the outer cover 40. Positioning the pad members 104,106 below at least one layer of material may provide various advantages in some aspects of the present invention. For instance, locating the pad members 104, 106 below at least one layer of material within the chassis 32 protects the pad members during shipping and storage and from forming a short circuit during use especially if the pad members are located adjacent one another. Another benefit to placing the pad members under at least one layer of material is the ability to easily manufacture the sensing article 20 at high machine speeds.

It should be understood, however, that in other aspects of the present invention the conductive pad members 104, 106 may be positioned at an exterior surface of the chassis 32. For instance, the pad members 104, 106 may be positioned on the outside surface or on the inside surface as desired.

Further details related to the structure, features, and materials of the absorbent article 20 may be found in co-pending and co-assigned U.S. patent application Ser. No. 11/414,032, filed on Apr. 27, 2006 by Allen, et al. and titled “An Array of Wetness Sensing Articles”; which is incorporated herein by reference to the extent it is consistent (i.e., not in conflict) herewith.

Referring to FIG. 1 for exemplary purposes, a communication system 110 (as depicted by ref. numerals 112 and 114) is shown attached to the conductive pad members 104, 106. As shown, in this aspect of the present invention, the communication system 110 generally includes a transmitter 112 and a receiver module 114. The transmitter 112 includes a pair of opposing terminals that are electrically connected to the corresponding conductive elements. When a body fluid is present in the sensing article 20, the open circuit formed by the conductive elements 100, 102 is closed which, in turn, activates the communication system 110. In particular, in this aspect of the present invention, the transmitter 112 sends a wireless signal to the receiver module 114 which then indicates to the home automation network 10 that a body fluid is present in the sensing article 20. In other various aspects of the present invention, any chemical or physical reaction that can change conductivity can also be sensed by this type of system—examples include conductivity changes associated with electrochemical sensors inside of the absorbent article 20 such as those described in co-pending and co-assigned U.S. patent application Ser. No. 11/314,438, filed on Dec. 21, 2005 by Ales, et al. and titled “Personal Care Products with Microchemical Sensors for Odor Detection”; which is incorporated herein by reference to the extent it is consistent (i.e., not in conflict) herewith. In other aspects of the present invention, the transmitter 112 may be embedded in or otherwise manufactured as a part of the absorbent article 20.

The transmitter 110 can also emit an audible signal or a visual signal to indicate to the user that the circuit has been closed. The audible signal, for instance, may be as simple as one or more beeps to perhaps emitting a musical tune. Similarly, if the transmitter 110 issues a visible signal, the visible signal may comprise one light, a few lights, or an interactive display.

In various aspects of the present invention, the sensing article 20 may include additional features such as those disclosed in co-pending and co-assigned U.S. patent application Ser. No. 11/303,283, filed Dec. 15, 2005 by Long, et al. and entitled “Garments With Easy-To-Use Signaling Device”; and U.S. patent application Ser. No. 11/215,937, filed Aug. 31, 2005 by Ales, et al. and entitled “Method of Detecting the Presence of an Insult in an Absorbent Article and Device for Detecting the Same”; which are incorporated herein by reference to the extent they are consistent (i.e., not in conflict) herewith. For example, the sensing article may also include other wetness sensing features such as fading ink, appearing ink, a wetness liner, or a cooling component.

The sensing article 20 may be a part of a wetness sensing system such as those described in above-referenced co-pending and co-assigned U.S. patent application Ser. No. 11/414,032, filed on Apr. 27, 2006 by Allen, et al. and titled “An Array of Wetness Sensing Articles.”

In other aspects of the present invention, the sensing article 20 may be constructed similarly to that described above except that the conductive elements 100, 102 are replaced by a biosensor 150.

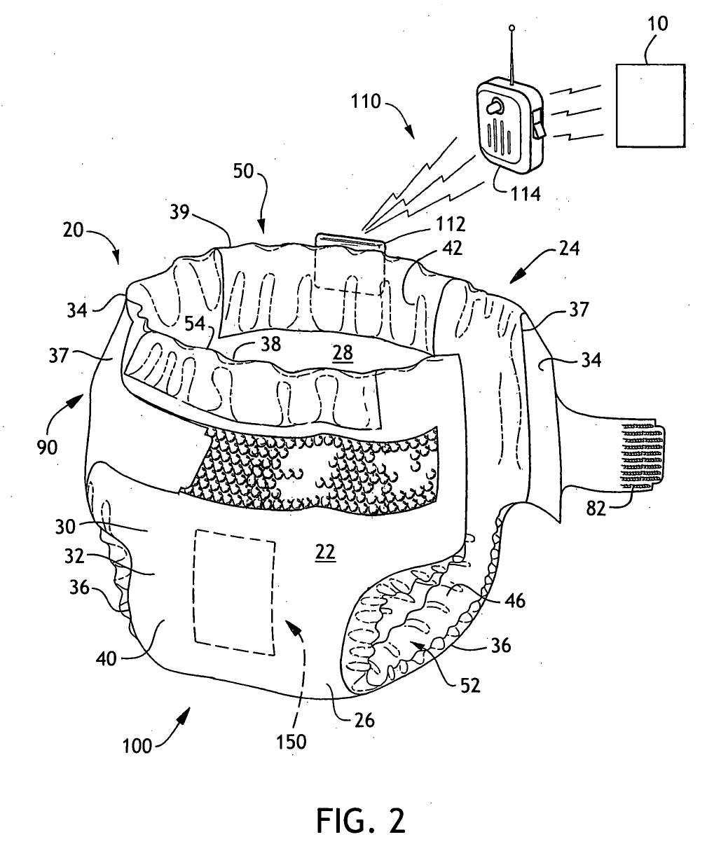

FIG. 2 shows additional details associated with a biosensor 150 in a sensing article 20. A biosensor 150 interacts with the wearer of the sensing article 20 to yield an analyte measurement conveyed to the transmitter 112, which in turn transmits the biosensor signal to the receiver module 114. The biosensor signal is then used by the home automation network 10 as described above. Additional detail related to the use of biosensors in absorbent articles is described in the previously-referenced U.S. patent application Ser. No. 10/277,170 by Lindsay et al. and titled “Healthcare Networks With Biosensors”; which is incorporated herein by reference to the extent it is consistent (i.e., not in conflict) herewith.

Biosensors 150 tied to a home automation network 10 may be used for numerous purposes, including:

detecting the onset of infection or the status of an infection for a recovering patient;

monitoring the health of fetus or mother during pregnancy (pregnancy management), detecting such things as premature delivery by monitoring uterine contractions, antiphospholipid antibodies, fetal fibronectin proteins, and so forth; monitoring reproductive status (e.g., onset of ovulation or other factors associated with fertility);

other hormone detection (e.g., growth factors, thyroid, menopause-related ones, etc.)

detecting the onset of menstruation;

monitoring analytes associated with renal disease, including analytes in the blood or urine measured before, during, or after dialysis, and analytes measured in any body fluids at home or for patients not receiving dialysis,

detecting factors related to stroke, including D-dimer in the blood or other body fluids;

monitoring the effectiveness or presence of a pharmaceutical agent such as an antibiotic;

detecting an enzyme or other factor associated with heart disease to alert a patient and/or caregiver of a potential cardiovascular problem;

identifying rheumatoid arthritis by detecting type I collagen crosslinked N-telopeptides in urine;

monitoring cyanosis or circulatory disorders in newborns, diabetics, and so forth;

monitoring the onset of a sleep apnea episode, coupled with treatments to enhance sleep when needed;

tracking body position in a bed and applied pressure against the skin of the patient in order to prevent or care for bedsores (decubitus ulcers) and other ulcers or wounds, including the use of printed arrays of pressure detecting films that can serve as a sensor indicating pressure applied by the body to various points under the body;

tracking indicators of health by monitoring of body odors or analytes in the gas phase near the body, using electronic nose technology or other sensors;

using archived time histories of one or more analytes as a record for identification of sudden changes in the treatment of a subject that may be traceable to changes in personnel, medication, and the like, wherein the time history may serve as a tool in detecting malpractice or other problems, or in verifying (or refuting) claims made by the user regarding health status of the subject;

monitoring levels of taurine in the body or in a local region, including monitoring taurine levels in a non-human mammal such as a domestic cat;

urinary tract infection testing;

yeast infection, bacterial infection, or other forms of vaginitis, including pH imbalance;

UV exposure detection; and

monitoring health factors in neonates, such as bilirubin levels for jaundice detection.

The biosensor 150 may provide measurements in real time, measurements at periodic intervals (e.g., snapshots in time), time-averaged results, and the like. The biosensor 150 can be worn on the body or against the body. By way of example, it may be placed inside or on an absorbent and-or disposable article such as a bed pad, a diaper, a sanitary napkin, facial tissue, ostomy bag, tampon, disposable garment, incontinence product, and so forth. It can also be an electrode, optical device, or other instrument, preferably miniaturized, that can respond to health indicators from the subject's body. The biosensor 150 may detect one or more analytes directly. Any suitable biosensor technology can be used, including dielectrophoresis, free-flow electrophoresis, ATP bioluminescence, DEFT, impedance, LAL, ELISA and other immunoassay methods, pH measurement, optical diffraction-based techniques, agglutination techniques, chromogenic agars, and molecular imprinting for the real-time analysis. Analysis of the detected signal to assess the health of the subject can be based on comparison to fixed parameters or parameters that are adjusted over time.

In addition to the signal from the biosensor 150, any number of additional signals (not shown) may be received by the receiver module 114 or other module and sent to the home automation network 10. Such signals can be transmitted by any means such as UWB signals, AM or FM radiofrequency signals, direct wiring, the Internet, a modem, and the like. The additional signals can include readings from other sensors providing measurements of factors such as room temperature, light levels, the location of the subject via a signal from a Global Positioning System (GPS) device or other positioning means, information regarding medications received, operational status of therapeutic devices, the presence of others in the room, whether or not the individual is in bed (e.g., using a load sensor in the bed), and the like. In one embodiment, the presence of specified objects or persons near the subject can be detected by detection means and transmitted with or in addition to the biosensor signal to the receiver module 114 or to another module (not shown) for continuous monitoring of the well-being of the patient.

As used herein, the term “analyte” means an atom, ion, molecule, macromolecule, organelle, or cell, or, optionally, a mixture thereof, that is detected and measured. The term “analyte” also means a substance in a medium including, but not limited to molecules such as proteins, glycoproteins, antibodies, antigens, hemoglobin, enzymes, target molecules that bind to or react with specific enzymes or other proteins, metal salts, ions (e.g., hydrogen ions, hydroxy ions, sulfates, sulfonates, phosphates, nitrates, nitrites, or electrolytes such as sodium, potassium, lithium, or calcium ions), fatty acids, neurotransmitters, hormones, growth factors, cytokines, monokines, lymphokines, lipocalins, nutrients, sugars, receptors, nucleic acids, fragments of DNA or RNA, and pharmaceutical agents or derivatives or metabolites thereof. The term “analyte” also means structured elements such as macromolecular structures, organelles and cells, including, but not limited to cells of ectodermal, mesodermal, and endodermal origin such as cells, blood cells, neural cells, immune cells, and gastrointestinal cells, and also microorganisms, such as fungi, viruses, bacteria and protozoa, or characteristic compounds produced by the same. For example, in pH measurement, the analyte can be hydrogen ions and/or hydroxy ions. Some analytes indicate a possible disease condition by either a higher or lower than normal level.

As used herein, “medium” and “biological sample” can refer to any material that can contain an analyte to be measured. A medium or biological sample can be any body fluid, including blood or any of its components (plasma, serum, etc.), menses, mucous, sweat, tears, urine, feces, saliva, sputum, semen, urogenital secretions, gastric washes, pericardial or peritoneal fluids or washes, a throat swab, pleural washes, ear wax, hair, skin cells, nails, mucous membranes, amniotic fluid, vaginal secretions or any other secretions from the body, spinal fluid, human breath, gas samples containing body odors, flatulence or other gases, any biological tissue or matter, or an extractive or suspension of any of these.

As used herein, “biosensor” refers to any sensor that collects data about a biological or physiological process. Biosensors can include any probe, such as those including biological material, which measures the presence or concentration of analytes such as biological molecules, biological structures, microorganisms, etc., by translating a biochemical interaction with the probe into a physical signal. More specifically, the term can refer to the coupling of a biological material (for example, enzyme, receptor, antibody, whole cell, organelle) with a microelectronic system or device to enable rapid low level detection of various substances in body fluids, water, and air.

As used herein, “biosensor signal” refers to a quantitative or qualitative measurement reading provided by a biosensor, which, without limitation, can be in the form of any of the following:

electronic data, either a digital or analog signal (such as electrical current or a voltage generated directly by the biosensor or indirectly by another device in response to a biosensor reading) that can in turn result in a display on an output device or in data being transmitted to a computer;

a visual cue such as a color change or altered position of an indicator needle or other visible indication of qualitative or quantitative information on devices such as liquid crystal panels, LED arrays, “electronic paper,” or a visible computer display of text or a static or animated image;

a sound such as a beep, a synthesized voice, or a prerecorded message;

a temperature change induced by the biosensor;

a tactile, vibration, or pressure signal induced by the biosensor; or

any other suitable means of generating a signal to convey information about a measurement made by the biosensor.

Methods for incorporating biosensors in absorbent articles such as diapers or sanitary napkins include those disclosed in U.S. patent application Ser. Nos. 09/299,399; 09/517,441; 09/517,481; 09/342,784; 09/342,289; and in U.S. Pat. Nos. 6,186,991 and 5,468,236. Any of these can be adapted for use with the present invention.

Various illustrative and non-limiting examples of the present invention are described herein. One solution includes a training pant incorporating a wetness sensor and a wireless link to an X10 receiver that leads to an annunciation somewhere in the home that the pant is wet. A more sophisticated version includes the pant as an integral part of a home automation mesh network. With whole house coverage, such condition monitoring could be extended to location monitoring (i.e. has the child entered or left a room and should the parent be alerted). Another solution includes monitoring an enuretic condition. When a child wets in an enuretic product, an alarm that is integrated with the home automation network 10 could sound to notify the caregiver and/or the child. The home automation network 10 then responds by lighting the way automatically for the child as the child goes to the bathroom. In addition, an activity and/or tilt accelerometer may monitor when the child has moved from the bed to a standing position in response to a signal or an internal urge; the home automation network 10 could then respond to this and/or deactivate this or other signals based on the child's attention to the event.

Another solution includes position monitoring for infants and newborns. For parents concerned with their infant's position during sleep, a small monitor could relay the position of the child to the home automation network 10 and notify the parents if the infant were to roll into an undesired position. For temperature measurement and adjustment, heat flux sensors associated with the bodies of people in the house aid in determining their relative comfort level. If more heat is determined to be needed, the home automation network 10 can automatically adjust the HVAC in the room and/or house. For monitoring activity, accelerometers can be used to determine the relative activity of a resident in the home. Especially when used in conjunction with heart rate and respiratory monitors, an activity monitor can relay when a person is sleeping. As the sleep state is entered, the home automation network 10 can respond by lowering the lights in that area, reducing heat, turning on a fan, and ensuring all electronics are on or off as intended by the resident.

For wellness monitoring, physiological parameters such as heart rate, blood pressure, and metrics such as body weight may be archived using an integrated system such as that described herein. Risk and location monitoring may be accomplished by monitoring whether the subject is approaching something hazardous such as a stove or pool, or whether the subject has left the house unescorted.

In an exemplary use of the present invention, a caregiver puts a sensing article 20 in the form of a wetness sensing training pant on a subject and attaches a transmitter 112. When the subject insults the sensing article 20 with urine, the urine closes the circuit of the conductive elements 100, 102 and the transmitter 112, resulting in the transmitter sending a signal to a receiver module 114 electromagnetically or physically coupled to a home automation network 10. The home automation network 10 then notifies the caregiver that an insult has occurred, where the notification is by audible or visual indication, or by a wireless or wired signal sent to the caregiver. The home automation network 10 may also use the home's intercom system to notify the caregiver. The home automation network 10 may also provide instructions with respect to the optimum response to the signal and insult. The home automation network 10 may also, for example, turn on the lights in a bathroom convenient to the subject.

In addition, use of the sensing article 20 may be benefited by increasing the availability, awareness, consistency, and caregiver confidence of sensing articles 20 as described in co-pending and co-assigned U.S. patent application Ser. No. 11/414,035, filed on Apr. 27, 2006 by Ales, et al. and titled “Wetness-Sensing Absorbent Articles”; which is incorporated herein by reference to the extent it is consistent (i.e., not in conflict) herewith.

### EXAMPLE

A prototype system was constructed that would energize a 120V electrical outlet if a training pant was insulted. The following components were modified to construct this prototype: an EagleEye MS14A Indoor/Outdoor wireless X10 motion detector, an IBM Home Director Remote Module HD501, and a HUGGIES PULL-UP training pant with two strips of conductive foil installed. The motion detector was modified by removing the motion detection sensors and replacing that part of the circuit with a drain resistor and the foil strips in the training pant. A PDA was plugged into the remote module to give an indication of when the module was on or off. The charge light on the PDA would illuminate when the module was on. The illumination and dimming of the charge LED was in sync with a clunking noise from the remote module when the remote module's internal relay opened or closed. After ensuring that the remote module was off and not affected by motion, a small quantity of bottled water (less than 2 oz. by visual inspection) was poured onto the training pant in the vicinity of the conductive foil strips to which the modified motion detector was attached. The module responded with a loud clunk and the PDA charge light illuminated. These indications proved that the training pant insult was converted into a signal that was usable by X10 devices and therefore by a home automation network.

These and other modifications and variations to the present invention may be practiced by those of ordinary skill in the art, without departing from the spirit and scope of the present invention, which is more particularly set forth in the appended claims. In addition, it should be understood that aspects of the various aspects of the present invention may be interchanged either in whole or in part. Furthermore, those of ordinary skill in the art will appreciate that the foregoing description is by way of example only, and is not intended to limit the invention so further described in such appended claims.

What is claimed:

1. A sensing system for use in conjunction with a home automation network controlling an environmental variable in a home, the system comprising:
a transmitter adapted to be used with an absorbent article having a sensor, wherein the transmitter is adapted to communicate with the sensor and directly with the home automation network.

2. The system of claim 1, wherein the environmental variable is one of temperature; humidity; lighting; physical security including surveillance and the control of doors, windows, and shutters; sound including home entertainment systems; appliances; and intercoms.

3. The system of claim 1, wherein the sensor includes a biological sensor.

4. The system of claim 3, wherein the biological sensor senses one of the presence of a bodily exudate, a condition marker, a disease marker, and the fullness of the absorbent article.

5. The system of claim 1, wherein the sensor includes a location sensor.

6. The system of claim 1, wherein the sensor includes an activity sensor.

7. The system of claim 1, wherein the sensor includes a wetness sensor.

8. The system of claim 1, wherein the transmitter is adapted to communicate by wireless connection.

9. The system of claim 1, wherein the home automation network is adapted to communicate with a caregiver.

10. The system of claim 9, wherein the caregiver resides in the home.

11. The system of claim 1, wherein the absorbent article is one of a diaper, a pant, an incontinence product, and a feminine article.

12. A sensing system for use in conjunction with a home automation network controlling an environmental variable in a home, the system comprising:
an absorbent article including a sensor and a transmitter, wherein the transmitter is adapted to communicate with the sensor and directly with the home automation network.

13. The sensing system of claim 12, wherein the sensor is a wetness sensor.

14. A physiological event notification system comprising:
an absorbent article including a physiological event sensor;
a home automation network controlling an environmental variable in a home; and
a transmitter associable with the absorbent article and in signal communication with the physiological event sensor and with the home automation network.

15. The physiological event notification system of claim 14, wherein the physiological event sensor is a wetness sensor.

16. A method for benefiting a caregiver, the method comprising:
providing an absorbent article including a biological sensor and a transmitter, wherein the transmitter is adapted to communicate with the biological sensor and directly with a home automation network; and
providing instructions to the caregiver with respect to responding to the home automation network once the home automation network has received a communication from the transmitter.

17. The method of claim 16, wherein the home automation network is adapted to respond to a communication from the transmitter.

18. The method of claim 16, wherein the home automation network provides instructions to the caregiver in response to the communication from the transmitter.

19. A sensing absorbent article system comprising:
an absorbent article having a liner, an outer cover, and an absorbent structure positioned between the liner and the outer cover, the absorbent article including a sensing means; and
a transmitter capable of communicating with the sensing means and a home automation network.

20. The sensing absorbent article system of claim 19, wherein the sensing means is a wetness sensor.
